(19) United States
(12) Patent Application Publication
(10) Pub. No.: US 2007/0265603 A1
(43) Pub. Date: Nov. 15, 2007

(54) Apparatus for correcting presbyopia

(76) Inventor: Roberto Pinelli, Brescia (IT)

Correspondence Address: KIRSCHSTEIN, OTTINGER, ISRAEL;& SCHIFFMILLER, P.C., 489 FIFTH AVENUE, NEW YORK, NY, 10017, US

(21) Appl. No.: 11/702,702

(22) Filed: Feb. 6, 2007

Related U.S. Application Data
(63) Continuation-in-part of application No. PCT/EP05/06736, filed on Jun. 22, 2005.

(30) Foreign Application Priority Data
Aug. 8, 2004 (IT) MI2004A-001625

(51) Int. Cl. A61B 18/20

(52) U.S. Cl. 606/5

(57) ABSTRACT

The apparatus for correction presbyopia comprises a photorefractive surgery laser beam device operating by means of a control program capable of managing the following instructions: extent of at least a first circular area of intervention, which determines at least first laser beam aiming parameters; at least a first depth of intervention in the first area of intervention, which determines at least first intensity and/or duration parameters for the laser beam; at least a first activation of the laser beam with the first aiming parameters an the first intensity and/or duration parameters; extent of at least a second circular area of intervention, concentric, superimposed and with a diameter different from the first area of intervention, which determines at least second laser beam aiming parameters; at least a second depth of intervention in the second area of intervention, which determines at least second intensity and/or duration parameters for the laser beam; and at least a second activation of the laser beam, following on from the first activation, with the second aiming parameters and the second intensity and/or duration parameters.

### CROSS REFERENCE TO THE RELATED APPLICATIONS

This application is a continuation-in-part of International Application No. PCT/EP2005/006736, designating the United States.

### TECHNICAL FIELD OF THE INVENTION

The present invention relates to an apparatus for correcting presbyopia.

### BACKGROUND ART

Vision defects (refraction defects) such as myopia, hypermetropia, astigmatism and presbyopia are traditionally corrected by means of lenses fitted to spectacles or by means of contact lenses.

With the development of knowledge in the field of eye surgery and with the introduction of diagnostic and surgical instruments of various types, among which the excimer laser and the microkeratome have played a leading role, refractory surgery has truly become a branch of ophthalmology and a specialism which now makes it possible to reduce considerably, and in most cases eliminate, the use of artificial optical devices (spectacles and contact lenses) to correct myopia, hypermetropia and astigmatism. This has become possible because of the more sophisticated techniques of refractive surgery, which are capable of altering the curvature of the anterior surface of the eye (cornea).

The various vision defects (myopia, hypermetropia and astigmatism) have their origin in conditions of the cornea which prevent images from being focused on the retina and for this reason do not permit good visual acuity such as is present in emmetropic individuals (who have no vision defects). Depending on whether a patient has myopia, hypermetropia, astigmatism or a combination of defects (hypermetropic astigmatism, myopic astigmatism), the corneal surface is too curved or too flat or has irregularities in the curvature of the meridians. Each of these conditions prevents images from being brought into focus on the retina and consequently produces distorted vision.

Many instruments used in refractive surgery are intended for altering the corneal curvature, thus remedying defective vision.

At present, on the other hand, no photorefractive surgery equipment is capable of correcting presbyopia, a defect which affects almost all of the population and unlike the common refraction defects is a malfunction of the crystalline lens and of the accommodation muscles, resulting from the normal ageing process.

This is because bringing a near object into focus is the result of the action of the ciliary muscle which, by contracting, acts upon the elasticity of the crystalline lens, causing it to assume a more or less spherical shape depending on the distance from the object to be brought into focus. Accommodation capacity decreases progressively with advancing age, from 40/45 years onwards, and changes up to around 65/70 years.

Consequently, presbyopia is generally corrected only by means of contact lenses or lenses fitted to spectacles, but this does of course tie the user to daily use of a particular artificial corrective device, which is sometimes inconvenient and approximate in correction.

### DISCLOSURE OF THE INVENTION

The technical problem addressed by the present invention is therefore to produce an apparatus for correcting presbyopia which eliminates the technical disadvantages of the prior art.

Within the scope of this technical problem, a purpose of the invention is to produce an apparatus for correcting presbyopia which frees the user from the need for daily use of a particular artificial device.

Another purpose of the invention is to produce an apparatus for correcting presbyopia which offers extremely effective and accurate correction.

The technical problem is solved and also these and other purposes are achieved according to the present invention by producing an apparatus for correcting presbyopia according to claim 1.

Other characteristics of the present invention are moreover defined in the subsequent claims.

### BRIEF DESCRIPTION OF THE DRAWING

FIGS. 1-3 depict a cornea irradiated by a laser beam for a piano profile, hyperoptic profile, and a myoptic profile, respectively.

### BEST MODES FOR CARRYING OUT THE INVENTION

Other characteristics and advantages of the invention will become clear from the description of a preferred but not exclusive embodiment of the apparatus for correcting presbyopia according to the invention, given purely by way of non-limiting example in the following examples.

The apparatus for correcting presbyopia operates on the basis of the assumption that the cornea is capable of using various points of focusing for distant and near vision, and that by altering the curvature in a differentiated manner, taking account of any defects in the patient's vision, and at the same time considering the characteristics of the cornea and of the vision system, and also the evolution of the presbyopia process, it is possible to operate on the cornea to alter its curvature at various points by multifocal treatment. Thus a laser device for photorefractive surgery is arranged which takes account of vision parameters such as the quality and degree of the vision defect, thickness of the cornea, identification of the optical area, dominance and non-dominance etc., and the age of the patient.

The apparatus for correcting presbyopia therefore comprises a photorefractive surgery laser beam device operating by means of a device control program capable of managing the following instructions:
extent of at least a first circular area of intervention, which determines at least first parameters for aiming the laser beam;
at least a first depth of intervention, in particular constant, in the first area of intervention, which determines at least first intensity and/or duration parameters for the laser beam;
at least a first activation of the laser beam with the first aiming parameters and the first intensity and/or duration parameters;
extent of at least a second circular area of intervention, concentric, superimposed and of different diameter, in particular larger, compared with the first area of intervention, which determines at least second parameters for aiming the laser beam;
at least a second depth of intervention, in particular constant, in the second area of intervention, which determines at least second intensity and/or duration parameters for the laser beam; and
at least a second activation of the laser beam, following on from the first activation, with the second aiming parameters and the second intensity and/or duration parameters.

The control program also executes the following instructions:
extent of at least a third circular area of intervention, concentric, superimposed and of smaller diameter than the first area of intervention, which determines at least third parameters for aiming the laser beam;
at least a third depth of intervention, in particular constant, in the third area of intervention, which determines at least third intensity and/or duration parameters for the laser beam; and
at least a third activation of the laser beam, following on from the first and second activation, with the third aiming parameters and the third intensity and/or duration parameters.

In detail, each area of intervention corresponds to an optical area of the cornea, while each depth of intervention corresponds to a depth of ablation of the corneal tissue.

Moreover, each area of intervention is extended by a corresponding circular marginal transition area in which the depth of intervention decreases gradually until it becomes zero.

Each marginal transition area in particular has an extent proportional to the corresponding area of intervention.

Each area of intervention corresponds to an optical area centred on the centre of the cornea and with a diameter greater than that of the maximum pupil dilation.

With reference to examples 1, 2 and 3 which follow, some possible methods are indicated for controlling the laser of the apparatus for correcting presbyopia.

### EXAMPLE 1

Emmetropic patient (with good normal distant vision) suffering from presbyopia (+2; age between 50 and 60 years).

Sequence of machine instructions:
key in data relating to the extent of the area of intervention: 6 mm;
key in data relating to the first depth of intervention: such as to reduce the presbyopic defect by +1;
first activation of the laser beam (corresponding to the execution of a first ablation over the whole optical area considered);
key in data relating to the extent of the second area of intervention: 6.5 mm;
key in data relating to the second depth of intervention: such as to reduce the presbyopic defect by another +1;
second activation of the laser beam (corresponding to the execution of a second ablation over the whole 6.5 mm optical area);

Correction of the induced myopia defect of 2 dioptres:
key in data relating to the extent of the third area of intervention: 5.2 mm;
key in data relating to the third depth of intervention: such as to reduce the myopic defect by −1;
third activation of the laser beam (corresponding to the execution of a third ablation over the whole 5.2 mm optical area);
key in data relating to the extent of the fourth area of intervention: 5.4 mm;
key in data relating to the fourth depth of intervention: such as to reduce the myopic defect by another −1;
fourth activation of the laser beam (corresponding to the execution of a fourth ablation over the whole 5.4 mm optical area).

### EXAMPLE 2

Myopic patient (−2 dioptres) also suffering from presbyopia (+2.5; age between 60 and 70 years)

Sequence of machine instructions:
key in data relating to the extent of the first area of intervention: 5.6 mm;
key in data relating to the first depth of intervention: such as to reduce the myopic defect by −1;
first activation of the laser beam (corresponding to the execution of a first ablation over the whole optical area considered);
key in data relating to the extent of the second area of intervention: 5.8 mm;
key in data relating to the second depth of intervention: such as to reduce the myopic effect by another −1;
second activation of the laser beam (corresponding to the execution of a second ablation over the whole 5.8 mm optical area);

Correction of the presbyopic defect:
key in data relating to the extent of the third optical intervention area: 6 mm;
key in data relating to the third depth of intervention: such as to reduce the presbyopic defect by +1.25;
third activation of the laser beam (corresponding to the execution of a third ablation over the whole 6 mm optical area);
key in data relating to the extent of the fourth area of intervention: 6.5 mm;
key in data relating to the fourth depth of intervention: such as to reduce the presbyopic defect by another +1.25;
fourth activation of the laser beam (corresponding to the execution of a fourth ablation over the whole 6.5 mm optical area).

Correction of the induced myopia of 2.5 dioptres:
key in data relating to the extent of the fifth optical intervention area: 5.2 mm
key in data relating to the fifth depth of intervention: such as to reduce the myopic defect by −1.25;
fifth activation of the laser beam (corresponding to the execution of a second ablation over the whole 5.2 mm optical area);
key in data relating to the extent of the sixth optical intervention area: 5.4 mm
key in data relating to the sixth depth of intervention: such as to reduce the myopic defect by another −1.25;
sixth activation of the laser beam (corresponding to the execution of a sixth ablation over the whole 5.4 mm optical area);

### EXAMPLE 3

Patient with hypermetropia of +1 and suffering from presbyopia (+1; age between 40 and 45 years)

Sequence of machine instructions:
key in data relating to the extent of the first optical intervention area: 6 mm;
key in data relating to the first depth of intervention: such as to reduce the hypermetropic defect by +1;
first activation of the laser beam (corresponding to the execution of a first ablation over the whole 6 mm optical area);
key in data relating to the extent of the second area of intervention: 6.5 mm;
key in data relating to the second depth of intervention: such as to reduce the presbyopic defect (+1);
second activation of the laser beam (corresponding to the execution of a second ablation over the whole 6.5 mm optical area);

Correction of the induced hypermetropia defect:
key in data relating to the extent of the third area of intervention: 5.2 mm;
key in data relating to the third depth of intervention: such as to reduce the hypermetropic defect (+1);
third activation of the laser beam (corresponding to the execution of a third ablation over the whole 5.2 mm optical area);

The instructions described above for each step (the operating sequences) may be copied to a magnetic medium capable of being decoded and interpreted by the computerized automatic system of the laser device.

The apparatus for correcting presbyopia designed in this way may undergo numerous changes and be produced in numerous variants, all of them falling within the scope of the inventive concept; moreover, all parts may be replaced by technically equivalent items.

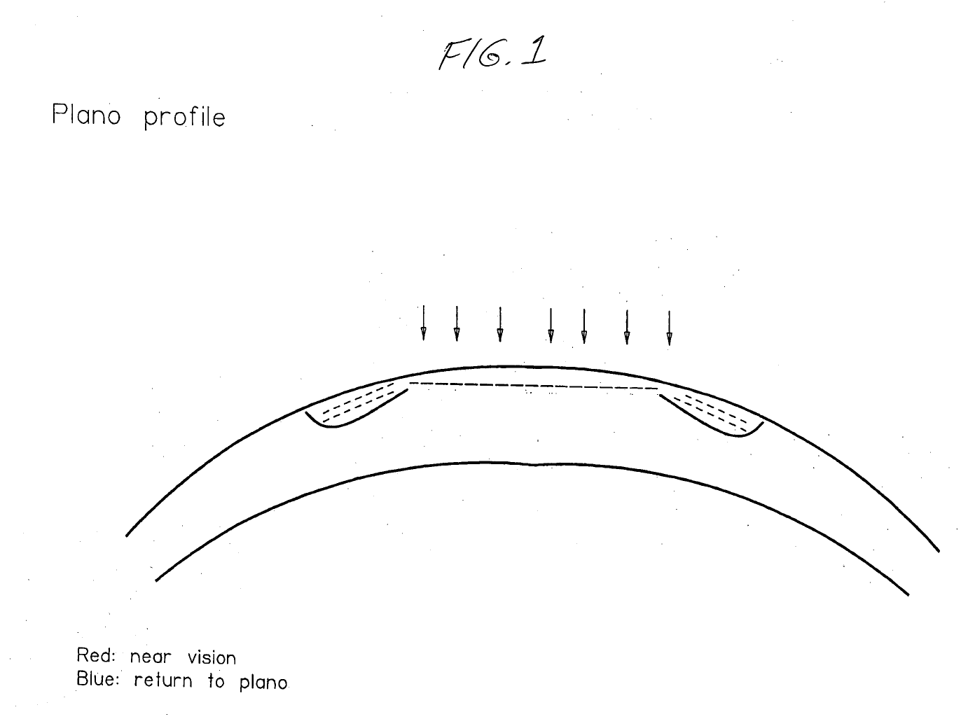

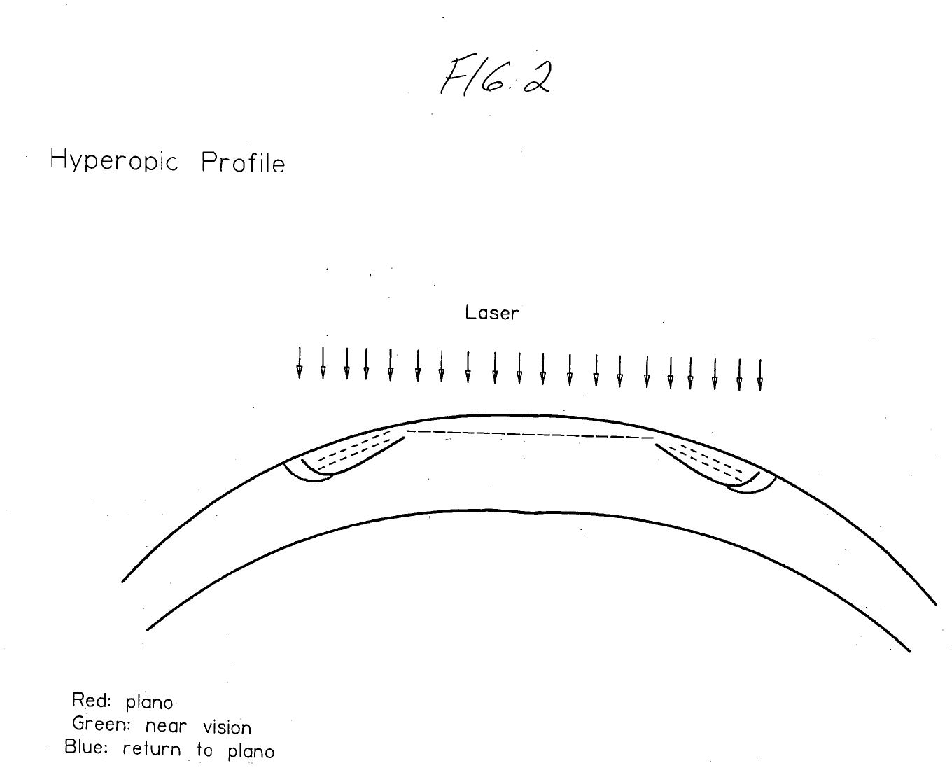

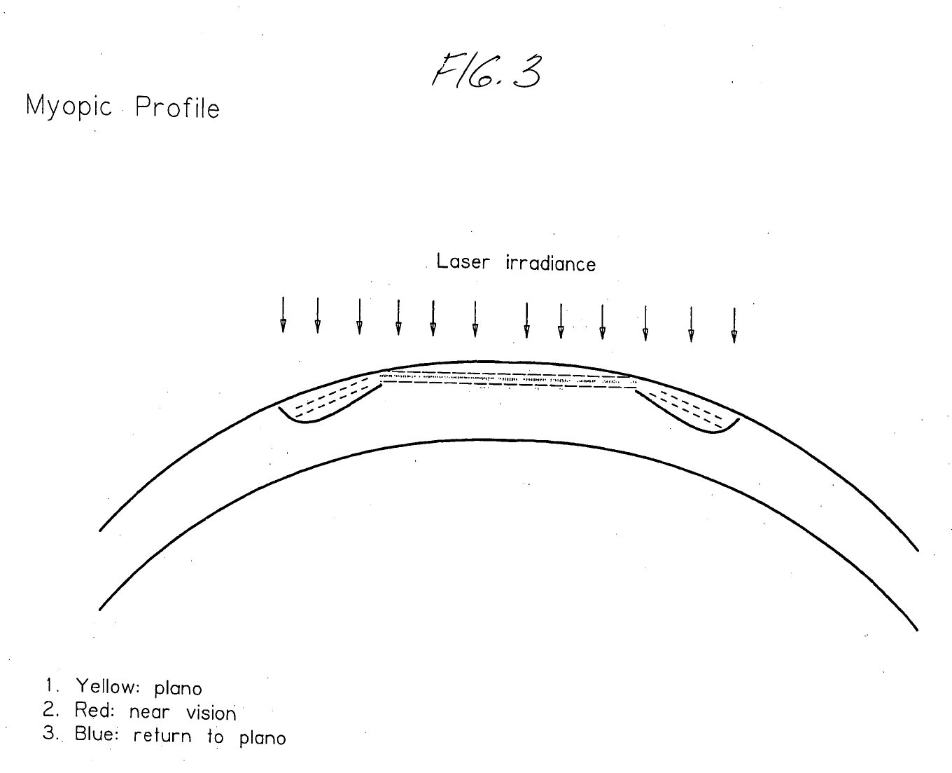

FIGS. 1-3 depict a cornea irradiated by a laser beam for a piano profile, hyperoptic profile, and a myoptic profile, respectively.

1-10. (canceled)

11: A method of correcting presbyopia by operating a photorefractory surgery laser beam device to emit a laser beam by means of a program controlling said device, comprising the steps of:
determining an extent of at least a first circular area of intervention, which determines at least first parameters for aiming said laser beam;
determining at least a first depth of intervention in said at least first area of intervention, which determines at least first intensity and/or duration parameters for said laser beam;
activating at least a first activation of said laser beam with said first aiming parameters and said first intensity and/or duration parameters;
determining an extent of at least a second circular area of intervention, concentric, superimposed and with a diameter different from said first area of intervention, which determines at least second parameters for aiming said laser beam;
determining at least a second depth of intervention in said at least second area of intervention, which determines at least second intensity and/or duration parameters for said laser beam; and
activating at least a second activation of said laser beam, following on from said first activation, with said second aiming parameters and said second intensity and/or duration parameters.

12: The method of correcting presbyopia according to claim 11, characterized in that said at least first and respectively at least second parameters for aiming said laser beam are determined by at least a first and respectively at least a second corneal optical area.

13: The method of correcting presbyopia according to claim 11, characterized in that said at least first and respectively at least second intensity and/or duration parameters are determined by at least a first and respectively a second depth of ablation of the corneal tissue.

14: The method of correcting presbyopia according to claim 11, characterized in that said at least first and respectively at least second intensity and/or duration parameters are determined by at least a first and respectively at least a second constant ablation depth.

15: The method of correcting presbyopia according to claim 13, characterized in that said at least first and respectively at least second intensity and/or duration parameters are determined by at least a first and respectively at least a second circular marginal area of transition of said at least first and respectively at least second optical area in which said at least first and respectively at least second depth of ablation decrease gradually until they reach zero.

16: The method of correcting presbyopia according to claim 12, characterized in that said at least first and respectively at least second intensity and/or duration parameters are determined by at least a first and respectively at least a second circular marginal area of transition of said at least first and respectively at least second optical area, proportional in extent to said at least first area and respectively at least second optical area, in which said at least a first and respectively at least a second depth of ablation decrease gradually until they reach zero.

17: The method of correcting presbyopia according to claim 11, characterized in that said at least first and at least second aiming parameters are determined by said at least a first optical area with a diameter less than said at least a second optical area.

18: The method of correcting presbyopia according to claim 11, characterized in that said first and respectively second aiming parameters are determined by a first and respectively a second optical area centered on the center of the cornea and with a diameter greater than that of the maximum pupil dilation.

19: The method of correcting presbyopia according to claim 12, and further comprising:
determining an extent of at least a third circular optical area, concentric, superimposed and with a diameter less than said first optical area, which determines at least third parameters for aiming said laser beam;
determining at least a third ablation depth in said at least a third optical area, which determines at least third intensity and/or duration parameters for said laser beam; and
activating at least a third activation of said laser beam, following on from said first and second activation, with said third aiming parameters and said third intensity and/or duration parameters.

20: The method of correcting presbyopia according to claim 11, and storing instructions on a medium readable by said laser beam device.
